(19) United States
(12) Patent Application Publication
(10) Pub. No.: US 2006/0105068 A1
(43) Pub. Date: May 18, 2006

(54) Dietary supplement formulations containing Hoodia gordonii

(76) Inventor: Albert M. Fleischner, Westwood, NJ (US)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 11/332,865

(22) Filed: Jan. 17, 2006

Related U.S. Application Data
(63) Continuation-in-part of application No. 10/693,442, filed on Oct. 24, 2003.

(51) Int. Cl. A61K 36/33

(52) U.S. Cl. 424/767

(57) ABSTRACT

A diet composition for weight control including effective amounts of hoodia gordonii cactus (whole plant/less roots), alone or together with any or all of chromium, vanadium amino acid chelate, glucomannan, sodium carboxymethylcellulose, citrus naringinine, green tea, cocoa extract, glucosamine HCl, ma huang, 3-acetyl-7-oxo-dehydroepiandrosterone, and coleus forskohlii. The effective amounts may be administered before each meal.

### RELATED APPLICATIONS

This application is a continuation-in-part of Ser. No. 10/693,442 filed 23 Oct. 2003.

### GOVERNMENT INTEREST

None.

### BACKGROUND

My invention relates to the evolving science that the Hoodia gordonii cactus (genus Trichocaulon), preferably in the form of sun-dried chips or 80 mesh powder, alone or combined with a blend of beneficial herbs and compounds, can benefit weight loss or weight control.

Approximately 97 million Americans, 61%, are overweight or obese. Individuals who are obese have at least a 50 percent increased risk of premature death. According to the United States Surgeon General, overweight and obesity are increasing. Obesity is a direct causal contributor to many diseases and exacerbates many others. Among these diseases are five of the leading causes of death in the West: stroke, atherosclerosis, cardiovascular disease, diabetes, and cancer.

A panel of experts chosen by the World Health Organization (WHO) in 1997 said that “Obesity's impact is so diverse and extreme that it should now be regarded as one of the greatest neglected public health problems of our time. It has an impact on health, which may well prove to be as great as that of smoking.” Obesity and health problems related to overweight are responsible for 6% of American health spending. In 2000, the economic cost of obesity in the United States was over $115 billion. The American Heart Association recently reclassified obesity as a major, modifiable risk and independent predictor for coronary heart disease. Obesity is associated with a higher risk of early death, diabetes, hypertension, and hyperlipidemia.

Overweight or obese people can suffer from osteoarthritis as a result of the extra pressure on the joints in the knees, hips, and lower back. Another medical problem that can be associated with obesity is gout, joint pain caused by excess uric acid. Sleep apnea, a breathing problem that causes interrupted breathing during sleep, is more common in overweight and obese people than in those who maintain healthy weight.

Besides the plethora of physical health problems that can be caused by obesity, there are numerous negative psychological and social effects of being overweight, such as depression and discrimination. One of the most difficult aspects of overweight and obesity may be the emotional suffering it can cause. Our society places great importance on physical attractiveness, and often equates thinness with being attractive. Obese people may face ridicule or discrimination at work, school, and in social situations. Feelings of rejection, shame, and depression are common.

Caloric reduction and increased exercise are the most often used therapies to combat obesity. However, this approach has a low success rate; individuals enrolled in weight loss programs lose 10% of their weight, but 35% to 65% is gained back within a year. It is therefore vital to identify factors affecting these negative weight loss outcomes and develop new treatments for obesity.

I have found that the plant hoodia gordonii may be effective in combating this problem.

The Hoodia gordonii cactus has been used safely and effectively for decades to temporarily stave off hunger and thirst. The San bushmen in South Africa ate the cactus to prevent hunger while hunting. This same plant has the potential to help Americans lose weight by suppressing the appetite over time.

Hoodia gordonii is a cactus. It has been used for years by the San tribesmen in South Africa to temporarily prevent hunger during extended hunting expeditions, during which food might not have been readily available. This use occurred as early as 1937, when a Dutch anthropologist studying the San noted their use of the Hoodia cactus.

While the cactus was used by the San for the temporary relief of hunger, such temporary or opportunistic use appears in fact to cause a long-term increase in body mass. VAN HEERDEN et al., U.S. Pat. No. 6,376,657, teaches administering sap and other extracts of Hoodia gordonii to laboratory rats. VAN HEERDEN teaches that administering Hoodia sap to laboratory rats increases basal food intake. The effect of a methanol extract of trichocaulon piliferum on rats' rate of food intake is shown in VAN HEERDEN at FIG. 2. FIG. 2 shows rats' basal rate of food intake (shown at days 3-5) remains roughly constant at 17 grams per day; after administering sap from the cactus (at day 5), however, the rats' rate of food intake decreases quite sharply for two days, and then rises to a rate of about 20 grams per day, a rate greater than the original basal rate.

The net effect of this change in eating rate is shown in FIGS. 5 and 6. FIG. 5 compares the body mass of control rats and rats fed various amounts of Hoodia sap. Net change in body mass over the two week study period is shown in FIG. 5; the control rats (Group 5 and 9) experienced moderate or significant net decreases in body mass, losing 18.91% and 3.51% of body mass, respectively. (This could be because the rats lacked adequate sleep or physical activity during the fourteen day test, or disliked the food they were given, etc. . . . ).

In contrast, rats fed hoodia sap (Groups 1, 2, 3, and 4) lost significantly less body mass than control Group 5. That is, they retained more body mass during the two week period. Curiously, the lowest dose (Group 1) and the higher doses (Groups 3 and 4) each had less effect than an intermediate dose (Group 2). Thus, while all groups showed less weight loss than control Group 5, the weight-retention effect may be potentially dose-dependent, or simply due to random error.

Similarly, rats fed spray-dried hoodia sap (Groups 6, 7, 8) lost more body mass than control Group 9, yet less than control Group 5.

This effect is somewhat clarified in VAN HEERDEN at FIG. 6. FIG. 6 shows that the control Group 5 experienced half of its total weight loss (10.45% of a total 18.91% weight loss) in the latter half (the second week) of the two-week experiment; this shows somewhat steady weight loss over time: roughly 9.5% per week. In contrast, control Group 9 experienced a 3.51% weight loss over two weeks, yet, as shown in FIG. 6, a 9.59% gain in the second week. This shows an 11.95% weight loss during the first week, followed by a 9.59% gain. This is significant because it indicates that the rats could experience routine weekly 10% weight fluctuations.

In contrast, rats administered spray-dried sap (Groups 6, 7, 8) showed net weight gain after sap administration. Rats administered sap (Group 1, 2, 3, 4) showed results comparable to the two controls, Groups 5 and 9.

I know of no similar body-mass measurement data from the San tribesmen. One may infer from this data that humans would have similar results—negligible to increased weight, compared to humans given no hoodia sap. In light of the teachings of VAN HEERDEN, I infer that the cactus was thus not used by the San tribesmen for weight loss. This is logical, because the San tribesmen did not, at the time, have any wide-spread obesity problem that I know of; to the contrary, one could assume that they might have had the opposite problem—that of struggling to obtain adequate food to survive.

VAN HEERDEN thus teaches that hoodia sap causes a net increase in body mass. To treat obesity, then, VAN HEERDEN teaches and claims the use not of hoodia, but of a specific chemical present in hoodia.

I advocate the opposite.

I believe that the hoodia plant itself, rather than a chemical extract of it, can be used to safely and effectively control obesity. My solution lies in the timing of the hoodia administration.

VAN HEERDEN replicated in laboratory rats the incidental (one-time) administration of hoodia. He shows that a one-time administration creates a transient appetite suppression phase (in his FIG. 2, with the amount used, perhaps 48 hours), followed by an appetite stimulation phase of indeterminate duration. I propose repeat administration, before the onset of the appetite stimulation phase. In other words, the administration occurs at least as frequently as the length of the appetite suppression phase. For example, VAN HEERDEN shows that with the amount of hoodia administered there, the appetite suppression effect is replaced by an appetite activating effect after about 48 hours; thus, with this amount of hoodia, I would require repeat hoodia administration at least once every 48 hours or, in any case, before the appetite-stimulating effect occurs.

I also advocate longer-term administration, repeated over a period of weeks or months. When this repeat-administration is practiced over an extended period of time (I expect at least 30-45 days), this may enable the user's body to adjust to a lower basal body weight, and thereby eventually perhaps eliminate the appetite stimulation phase altogether, and thus avoiding the eating binge—and weight gain—that follows incidental hoodia administration.

I prefer to administer hoodia together with various other ingredients, which I believe create some synergy in efficacy. That is, a person may require 10 mg of hoodia per day to effect long-term weight reduction; the same person, if also given a stimulant (e.g., caffeine, ephedra) or glucosamine (or both), may only need 5 mg of hoodia to achieve the same weight reduction.

I will thus first discuss these other ingredients, and then disclose my various currently-preferred specific combinations or formulae that combine these ingredients.

Chromium

Chromium is one of the sixteen essential trace minerals that help keep the body healthy and fit. It is the most important nutrient in controlling blood sugar and sugar cravings, and contains a wide variety of benefits. Chromium helps insulin metabolize fat, convert protein into muscle, and convert sugar into energy, thereby encouraging weight loss and increasing lean body mass. Chromium is essential for the metabolism of glucose into energy. Chromium deficiency can trigger a craving for sweets. Research suggests that chromium supplementation can cause improvements in fat-burning. It also plays a role in lowering harmful LDL cholesterol and increasing beneficial HDL cholesterol.

To be metabolized, chromium is bound to another substance. The most effective and safest form of chromium is niacin-bound chromium nicotinate (or polynicotinate), such as chromium chelavite. Picolinic acid, used in chromium picolinate, is not listed as Generally Regarded As Safe (GRAS) by the FDA; niacin is listed as GRAS. Furthermore, chromium-niacin complex is believed to be biologically active in improving insulin action. Chromium is an extremely beneficial natural product for metabolism, as long as the picolinic variety is avoided.

Vanadium Research at the Grand Forks Human Nutrition Research Center supports vanadium as an essential nutrient that is beneficial for thyroid hormone metabolism. Vanadium has several physiological insulin-like effects, making it likely to have a positive effect on carbohydrate metabolism and weight loss. Vanadium may also inhibit appetite and curb cravings. The decrease in body weight caused by vanadium can be largely ascribed to less food intake caused by taste aversion and a possible effect at the appetite center. Studies have also shown that the insulin-like properties of vanadium may benefit those with diabetes.

Glucomannan

Glucomannan is a dietary fiber derived from the root of the Amorphophallus Konjac. Fiber-containing foods are known to reduce cholesterol and improve constipation, but they also assist in weight loss by creating a feeling of satiety. Glucomannan is an extremely beneficial form of fiber in that only small amounts are needed to create a feeling of fullness. In water, glucomannan swells up to many times its original volume. Several scientific studies have corroborated glucomannan's value in causing weight loss.

Sodium Carboxymethylcellulose

This agent is used as a thickener, binder, emulsifier, and stabilizer. It is sometimes used as an antacid, but is most adaptable as a nontoxic, indigestible, unabsorbable, hydrophilic gel as a bulk laxative. Sodium Carboxymethylcellulose fills up the stomach and encourages a sense of satiety.

Citrus Naringinine

Naringinine is a powerful citrus extract that aids in weight loss. Naringinine inhibits weight loss barriers, curbs the appetite, and acts as a source of soluble fiber. It is also an antioxidant that prevents damage by free radicals in the body. Naringinines have been shown to inhibit enzymes called cytochrome P450; excessive levels of CYP450 are associated with obesity. A hormone from the thyroid gland, throxine, is involved in fat breakdown and metabolism. Research indicates a 60-80% reduction in CYP450 when throxine is increased.

Green Tea

Green Tea (camellia sinensis) provides a natural source of caffeine, which increases the metabolism and aids in weight loss. But the efficacy of green tea for weight loss is greater than can be attributed to its caffeine content per se; its thermogenic properties reside primarily in the interaction between its high content of catechin-polyphenols and caffeine with sympathetically released noradrenaline (NA). Green tea extract is effective in stimulating thermogenesis by relieving inhibition at different control points along the NA-cAMP axis. This synergistic interaction between catechin-polyphenols and caffeine to augment and prolong thermogenesis has value in assisting the management of obesity.

Scientists at the University of Chicago's Tang Center for Herbal Medicine Research found that epigallocatechin gallate, a substance derived from green tea, causes rats to lose up to 21% of their body weight. After seven days of injections, the rats showed a 60% decrease in appetite. (Kao Y H, et. al. Modulation of Endocrine Systems and Food Intake by Green Tea Epigallocatechin Gallate. Endocrinology. 141(3):980-7,2000.) Epigallocatechin gallate from green tea polyphenols significantly reduce food intake, body weight, cholesterol and triglycerides, as well as growth of the prostate, uterus, and ovary; it may interact specifically with a component of a leptin-independent control pathway. Green tea has thermogenic properties, promotes fat oxidation, and plays a role in the control of body composition via sympathetic activation of thermogenesis, fat oxidation, or both. Another study showed that green tea extract induced thermogenesis, causing weight loss in humans. (American Journal of Clinical Nutrition—University of Geneva, Switzerland (AR25, patented green tea extract)

Green tea extract is bioflavonoid-rich and potent. The polyphenols in green tea, especially the catechin component, offer antioxidant activity to fight free radicals. Catechins are water-soluble compounds that are easily oxidized. Green tea polyphenols have demonstrated significant antioxidant, anticarcinogenic, anti-inflammatory, thermogenic, probiotic, and antimicrobial properties in numerous human, animal, and in vitro studies.

Theobromine

Theobromine is a dimethylxanthine, in the same class of compounds as caffeine and theophylline. Xanthines occur naturally in about 60 different plants plants such as cocoa (from Cocoa leaves), tea, and coffee. Theobromine is the predominant dimethylxanthine in cocoa beans.

Theobromine affects humans in a similar way as caffeine, but on a smaller scale. It is a diuretic, a mild stimulant, and it relaxes the smooth muscles of the bronchi in the lungs. Theobromine has been employed as a diuretic because its action on the kidneys is longer-lasting than other xanthines. It acts by inhibiting reabsorption in the renal tubules. Cocoa extract is safe and practically free of toxicity.

Glucosamine Sulfate

Glucosamine is a patented ingredient that permits fat to be burned instead of stored, which results in weight loss. When food enters the body at a faster rate than energy is consumed, the cellular level of adenosine triphosphate rises. Cells, however, do not store this extra energy in this form. When adenosine triphosphate levels in cells rise, this inhibits glycolysis and allows glucose to be converted into fat and stored in the body. When stored body fat is broken down and used, lipase enzymes hydrolyze triglycerides into glycerol and free fatty acids during the breakdown of fat, called lipolysis. The free fatty acids bring energy to the organs for aerobic respiration.

The effect of insulin on the formation of fatty acids in the body is delayed by glucosamine, indicating that glucosamine plays a role as a messenger for this insulin effect. Insulin is secreted when the sugar content is high and allows for fat storage. Fat cells can't be metabolized when there are high insulin levels in the body. High insulin levels also trigger the hypothalamus to send hunger signals, which sets off carbohydrate cravings. Thus one eats more, which leads to even more insulin. These extra carbohydrates are converted into glucose and then stored in the body as fat.

Glucose triggers a rise in insulin. The insulin lowers and regulates blood glucose levels through many actions, one of which is lipogenesis, or the conversion of carbohydrates and proteins into fats. Fat can't be metabolized when insulin levels are high; when high insulin levels exist, lipolysis is blocked and fat is stored in the body. Lipolysis is necessary to supply the cellular energy source ATP, which is needed for contraction of muscles. When the level of insulin is reduced, fat is burned and weight decreases. Glucosamine delays the effect of insulin without significant adverse reactions. When used as directed, glucosamine is safe and promotes weight loss.

Ma Huang

Ma Huang or ephedra is a member of the Ephedracae family of herbs. It is perhaps the world's oldest medicine; it has been used in China for thousands of years to treat symptoms of asthma and upper respiratory infections. Chiang Chung-Ching (142-212 AD) treated asthma with ephedra. Zen monks used ephedra to encourage calm concentration during meditation. According to legend, ephedra tea was given to bodyguards of Genghis Khan to keep them from falling asleep on sentry duty. Early American settlers used ephedra, also called Mormon Tea or Squaw Tea, to treat headaches, fevers, colds and hay fever. Varieties of ephedra are also grown in Europe, India, Australia, Afghanistan, and in the dry Southwest of the United States. Compounds derived from ephedra are currently used in many over-the-counter cold and allergy medications.

Ephedra causes vasoconstriction of blood vessels, dilates the bronchial tubes, and stimulates the heart, which results in thermogenesis (the burning of fat). Ma huang has the ability to open up adrenergic receptor sites found primarily in the heart and lungs, thereby increasing metabolic rate and calorie consumption. The result is the release of fatty acids from stored fat cells and quicker consumption of fat into energy. At least 55 studies confirm ephedra's safety and efficacy in causing weight loss in overweight but otherwise healthy people.

Ephedra contains two alkaloids, ephedrine and pseudoephedrine. The main constituent, ephedrine, is a bronchodilator and stimulates the sympathetic nervous system. It has antispasmodic properties, acting on the air passages by relieving swelling of the mucous membrane. Pseudoephedrine is a nasal decongestant and has a weaker stimulating effect on the heart and blood pressure. Doctors use these alkaloids to treat bronchial asthma, bronchitis, emphysema, persistent coughs, wheezing and shortness of breath. Ma Huang can help break fevers, clear blocked sinuses, treat allergic skin reactions such as hives, relieve general body pain, and treat low blood pressure, rheumatism and narcolepsy.

Let us look at six significant studies which demonstrate why ephedra is effective and safe for weight loss.

Recently, a randomized, placebo-ontrolled clinical study by James Blum, Ph.D. and Peter Marshall, M. D., in which the protocol had IRB approval, was completed. The results demonstrated 92% of the participants had significant weight loss using an ephedra/caffeine-based weight reducing agent. After eight weeks, the ephedra/caffeine group lost 12.75 pounds, while the placebo group lost only 5.63 pounds. The study group also lost 6.28 percent body fat, while the placebo group lost 2.73 percent body fat. There were no major adverse events reported. This study has just been submitted for publication. (1)

Another study on ephedra and weight loss was conducted at the New York Obesity Research Center at St. Luke's-Roosevelt Hospital by Boozer et. al. In this study, 167 obese men and women were given either a combination of ephedra and caffeine or a placebo for six months, in doses of 90 mg. ephedrine alkaloids and 192 mg. of caffeine per day. The ephedra/caffeine group lost an average of 15.2 pounds, while the placebo group lost only 6.8 pounds. The ephedra/caffeine group also lost a significant amount of body fat compared with the placebo group; the ephedra/caffeine group lost 3.2% body fat, while the placebo group lost only 0.6% body fat. In this study, it was concluded that herbal ephedra and caffeine promoted weight loss and fat reduction, as well as improving blood lipids, without adverse events. (2)

Boozer et al. conducted research at St. Luke's-Roosevelt Hospital in which sixty-seven overweight subjects were given either placebo or 72 mg. ephedra/240 mg. caffeine (guarana) a day for the study period of eight weeks. The ephedra/caffeine group had significantly higher weight loss than the placebo group, 7.5+/−8.8 pounds compared to 1.75+/−5.3 lbs. The treatment group also had more fat loss than the placebo group, −2.1+/−3.0% compared to 0.2+/−2.3%. The herbal ephedra/caffeine mixture effectively promoted short-term weight and fat loss. (3)

The effects of ephedra/caffeine on weight loss were studied by Astrup et. al. at the Research Department of Human Nutrition at the Royal Veterinary and Agricultural University in Denmark. One hundred and eighty obese patients were put on a 1000 calorie/day diet and either an ephedrine/caffeine combination (20 mg/200 mg day), ephedrine alone (20 mg), caffeine alone (200 mg), or placebo, three times a day for eight weeks. Weight loss was significantly higher in the combination group than with placebo from week 8 through 24 (36.6+1-15.0 lbs vs. 29.1+/−14.55 lbs). Weight loss in both the ephedrine and caffeine groups was similar to that of placebo. The scientists concluded that the ephedrine/caffeine combination is useful for the treatment of overweight and obesity. (4)

Researchers in Denmark, Breum et. al., found that the combination of ephedrine and caffeine is more effective in weight loss than dexfenfluramine. One hundred and three obese patients were included in a fifteen week double-blind study. All subjects ate a 1200 calorie/day diet, supplemented by either 15 mg dexfenfluramine twice daily or 20 mg ephedrine/200 mg caffeine three times a day. Those in the herbal ephedrine/caffeine group lost significantly more weight than those in the dexfenfluramine group. After fifteen weeks, the dexfenfluramine group had lost 15.2+/−9.5 while the ephedra/caffeine group lost 18.3+1-11.5 lbs. (5)

Astrup and Toubro conducted research in Denmark that examined the thermogenic effects of ephedrine and caffeine, given alone and in combination. They concluded that the thermogenic effects of the combination of ephedrine and caffeine (20 mg/200 mg three times a day) was higher than either ephedrine or caffeine alone. The combination had pronounced effects on glucose metabolism and fat loss. The researchers found that the combination “exerted a supra-additive synergism on thermogenesis.” (6)

Almost all experts agree that ephedra, when combined with the correct amounts of caffeine, works in controlling weight. This combination herbal treatment works as well as, and in some cases better than, prescription weight loss products.

Despite recent debates concerning the safety of ephedra for weight loss, the herb enjoys a long history of safe use provided it is used properly. It should be noted that any product, including products that the American public accept as safe such as strawberries, peanuts, and aspirin, can be dangerous to certain people. Therefore it is vital that ephedra is used only by healthy people according to package directions. The safety of ephedra has been extensively reviewed by independent internationally recognized experts, including a former FDA toxicologist, several other pharmacologists and toxicologists with FDA expertise, and specialists in cardiology, epidemiology and other areas. This review has included ephedra with caffeine. The experts who have reviewed all the relevant historical and clinical data agree that ephedra is safe when used according to instructions.

Between 12-17 million people in America use ephedra products each year. In the past decade or so, there have been only two deaths, three myocardial infarctions, nine cerebrovascular accidents, three seizures, and five psychiatric cases identified as sentinel events with prior ephedra consumption, according to the government-sponsored RAND Corporation report. Considering the number of servings of ephedra used in the United States each year, these events are extremely rare. Note that 7,600 people die each year in the United States as a result of consuming aspirin and associated compounds in the therapeutic range.

Because ephedra stimulates the nervous system, it is a popular and effective weight loss aid. Ephedra suppresses the appetite and stimulates the thyroid gland, which stimulates metabolism by causing a fat-burning thermogenic effect.

### 3-acetyl-7-oxo-dehydroepiandrosterone (or “7-Keto”)

7-Keto is a natural derivative of DHEA that was discovered in human urine in 1958 and later confirmed in 1979. It is a non-glucocorticoid, non-mineralocorticoid, non-adrogenic C-19 steroid, a member of the androgen family. 7-Keto derivatives are not convertible to androgens, and do not activate the androgen receptor in human prostate cells. It does not increase testosterone levels, and there have been no reports of virilization effects in 9 controlled clinical trials or anecdotal reports. 7-Keto's downstream metabolites have been identified and their structures confirmed.

There are 22 published articles on 7-Keto that show its safety and efficacy. In these studies, no adverse effects were noted in vital signs or labs. Adverse advents and serum hormone levels did not differ between the treatment and placebo subjects, and all hormone levels remained within the normal ranges. In a study by the Minnesota Applied Research Center, the researcher found that 7-Keto caused statistically significant reductions in body weight, body mass index (BMI), and waist and hip circumference than placebo over an 8-week treatment period. 7-Keto was well tolerated and no serious adverse events were reported. (1) 7-Keto has the ability to enhance thermogenesis, and through that mechanism accelerates the use of fat stores for heat production. 7-Keto causes weight loss by enhancing the activity of three other enzymes: glycerol-3-phosphate dehydrogenase, malic enzyme and fatty acyl CoA oxidase.

The activation of glycerol-3-phosphate dehydrogenase causes an up-regulation of the glycerol-3-phosphate shuttle, which encourages the production of heat rather than ATP. When malic enzyme is activated, it is converted to pyruvate and NADPH in the soluble portion of the cytoplasm. There is then an excess of NADPH, which is transported into the mitochondria where it is converted to ATP and heat. The activation of fatty acyl CoA oxidase results in an enhancement of fatty acid breakdown, which produces more acetyl CoA, NADH and FADH2. This drives the cell to use the fatty acids for energy, promoting the breakdown of triglycerides.

The acetyl CoA, NADH and FADH2 are converted to ATP and heat at an unusually fast pace. All three of these enzyme activations push energy producing substrates to produce more heat than ATP; this is the biological definition of thermogenesis. These enzyme activations also promote the utilization of fat stores for energy and heat production, causing weight loss.

Coleus forskohlii

Coleus is a perennial plant in the mint family. Forskolin, a chemical found in coleus, activates an enzyme (adenylate cyclase) that affects every cell in the body and significantly influences metabolic processes. Forskolin can influence calcium concentration in cells, increase thermogenesis and lean body mass, and promote weight loss. The homeostasis of calcium concentration in cells is important for muscle contraction, secretory processes, hormone function, and to promote smooth functioning of vital body organs. Coleus aids in weight loss and maintenance of lean body mass due to its ability to break down stored body fat, as well as inhibit the synthesis of adipose tissue. It increases thyroid hormone production and release of fatty acids, thereby increasing metabolism. Coleus may also reduce inflammation and improve blood pressure and cardiac function.

Two recent studies suggest that coleus forskohlii has significant metabolic qualities.

In one study conducted in 1999, six overweight women were given 250 mg. of coleus forskolin extract two times daily for eight weeks. During the study, body weight and fat content decreased significantly, while lean body mass increased significantly. The average weight loss was about nine pounds. (21)

Dr. Richard Kreider, a respected sports nutrition professional, recently studied 23 overweight females who received 25 mg of forskolin two times a day for twelve weeks, while others received placebo. The forskolin group had a decrease in bodyweight while the placebo group experienced weight gain. Those in the forskolin group also reported more energy and decreased appetite. Dr. Kreider concluded that coleus may promote weight and fat loss and mitigate weight gain in overweight subjects. (22)

The existing information suggests that coleus may play a significant role in promoting weight loss. Coleus has been recognized as having similar effects as ephedra regarding its ability to break down stored body fat. There are currently two more clinical research studies underway regarding coleus for weight loss; the results of these studies have not yet been published.

Given these components, I have developed various combinations to offer various synergistic effects. Formula 1 is a general weight loss formula, to obtain the benefits of each component yet avoid the potential harmful side effect of having too much of any one component.

Formula 1

| | Mg per | | | |
|---|---|---|---|---|
| | tablet | | Ranges | |
| Chromium | 75 | mcg | 50-200 | mcg |
| (as chromium Chelavite ™ dinicotinate | | | | |
| glycinate) | | | | |
| Vanadium | 15 | mcg | 10-50 | mcg |
| (as vanadium amino acid chelate) | | | | |
| Glucomannan | 200 | mg | 0-400 | mg |
| Sodium Carboxymethylcellulose | 50 | mg | 25-200 | mg |
| Citrus naringinine | 5 | mg | 0-15 | mg |
| Glucosamine HCL | 50 | mg | 0-200 | mg |
| Or Glucosamine KCL | | | | |
| Cocoa Extract Standardized for PEA | 162.5 | mg | 0-500 | mg |
| (Phenylethylamine, | | | | |
| tyramine and 10% theobromine) | | | | |
| Green Tea Extract Standardized for ECGC | 125 | mg | 0-250 | mg |
| (epigallocatechin gallate | | | | |
| polyphenols and 40% | | | | |
| caffeine) | | | | |
| Hoodia gordonii cactus | 75 | mg | 5-200 | mg |
| (whole plant/less roots) | | | | |
| Plus suitable excipients | | | | |

A formula potentially suited for diabetic use, as it minimizes the adverse impact on insulin levels, is in Formula 2.

Formula 2

| | | | |
|---|---|---|---|
| Chromium (as chromium Chelavite ™ | 75 | mcg | 0.075 |
| dinicotinate glycinate) | | | |
| Vanadium (as vanadium amino acid | 15 | mcg | 0.015 |
| chelate) | | | |
| Sodium Carboxymethylcellulose | 100 | mg | 100.000 |
| Citrus naringinine | 7.50 | mg | 7.500 |
| Cocoa Extract Standardized for PEA | 162.50 | mg | 162.500 |
| (Phenylethylamine, tyramine and 10% | | | |
| theobromine) | | | |
| Green Tea Extract Standardized for | 250 | mg | 250.000 |
| ECGC | | | |
| (epigallocatechin gallate polyphenols | | | |
| and | | | |
| 40% caffeine) | | | |
| Hoodia gordonii cactus (whole | 100 | mg | 100.000 |
| plant/less roots) | | | |
| Plus suitable excipients | | | |

A higher-potency formula with minimal diabetic or hypertensive side-effects is given in Formula 3.

Formula 3

| | | | |
|---|---|---|---|
| Chromium (as chromium Chelavite ™ | 75 | mcg | 0.075 |
| dinicotinate glycinate) | | | |
| Vanadium (as vanadium amino acid | 15 | mcg | 0.015 |
| chelate) | | | |
| Sodium Carboxymethylcellulose | 100 | mg | 100.000 |
| Citrus naringinine | 7.50 | mg | 7.500 |
| Green Tea Extract Standardized for | 250 | mg | 250.000 |
| ECGC | | | |
| (epigallocatechin gallate polyphenols | | | |
| and | | | |
| 40% caffeine) | | | |
| Hoodia gordonii cactus (whole | 100 | mg | 100.000 |
| plant/less roots) | | | |
| Plus suitable excipients | | | |

A caffeine free formula is provided in Formula 4.

Formula 4

| | | | |
|---|---|---|---|
| Chromium (as chromium Chelavite ™ | 75 | mcg | 0.075 |
| dinicotinate glycinate) | | | |
| Vanadium (as vanadium amino acid | 15 | mcg | 0.015 |
| chelate) | | | |
| Glucomannan | 200 | mg | 200.000 |
| Sodium | 50 | mg | 50.000 |
| Carboxymethylcellulose | | | |
| Citrus naringinine | 5 | mg | 5.000 |
| Cocoa Extract Standardized for PEA | 325 | mg | 325.000 |
| (Phenylethylamine, tyramine and 10% | | | |
| theobromine) | | | |
| Hoodia gordonii cactus (whole | 100 | mg | 100.000 |
| plant/less roots) | | | |
| Plus suitable excipients | | | |

An alternative caffeine-free formula is provided in Formula 5.

Formula 5

| | | | |
|---|---|---|---|
| Chromium (as chromium Chelavite ™ | 150 | mcg | 0.150 |
| dinicotinate glycinate) | | | |
| Vanadium (as vanadium amino acid | 30 | mcg | 0.030 |
| chelate) | | | |
| Glucomannan | 400 | mg | 400.000 |
| Sodium Carboxymethylcellulose | 100 | mg | 100.000 |
| Citrus naringinine | 10 | mg | 10.000 |
| Hoodia gordonii cactus (whole | 150 | mg | 150.000 |
| plant/less roots) | | | |
| Plus suitable excipients | | | |

An alternative caffeine-free formula is provided in Formula 6.

Formula 6

| | | | |
|---|---|---|---|
| Chromium (as chromium Chelavite ™ | 150 | mcg | 0.150 |
| dinicotinate glycinate) | | | |
| Vanadium (as vanadium amino acid | 30 | mcg | 0.030 |
| chelate) | | | |
| Glucomannan | 400 | mg | 400.000 |
| Sodium Carboxymethylcellulose | 100 | mg | 100.000 |
| Citrus naringinine | 10 | mg | 10.000 |
| Glucosamine HCL Or Glucosamine | 100 | mg | 100.000 |
| KCL | | | |
| Hoodia gordonii cactus (whole | 150 | mg | 150.000 |
| plant/less roots) | | | |
| Plus suitable excipients | | | |

A mild appetite suppression formula suitable for use over extended periods of time 5 (weeks or months) without interfering with sleep cycle is provided in Formula 7.

Formula 7

| | | | |
|---|---|---|---|
| Chromium (as chromium Chelavite ™ | 75 | mcg | 0.075 |
| dinicotinate glycinate) | | | |
| Vanadium (as vanadium amino acid | 15 | mcg | 0.015 |
| chelate) | | | |
| Glucomannan | 200 | mg | 200.000 |
| Sodium | 50 | mg | 50.000 |
| Carboxymethylcellulose | | | |
| Citrus | 5 | mg | 5.000 |
| naringinine | | | |
| Glucosamine HCL | 100 | mg | 100.000 |
| Or Glucosamine KCL | | | |
| Cocoa Extract | 200 | mg | 200.000 |
| Standardized for PEA | | | |
| (Phenylethylamine, | | | |
| tyramine and 10% | | | |
| theobromine) | | | |
| Green Tea Extract | 165 | mg | 165.000 |
| Standardized for ECGC | | | |
| (epigallocatechin gallate polyphenols | | | |
| and | | | |
| 40% caffeine) | | | |
| Hoodia gordonii cactus | | | |
| (whole plant/less roots) | 7.5 | mg | 7.500 |
| Plus suitable excipients | | | |

An alternative mild appetite suppression formula suitable for use over extended periods of time (weeks or months) without interfering with sleep cycle is provided in Formula 8.

Formula 8

| | | | |
|---|---|---|---|
| Chromium (as chromium Chelavite ™ | 75 | mcg | 0.075 |
| dinicotinate glycinate) | | | |
| Vanadium (as vanadium amino acid | 15 | mcg | 0.015 |
| chelate) | | | |
| Glucomannan | 200 | mg | 200.000 |
| Sodium | 50 | mg | 50.000 |
| Carboxymethylcellulose | | | |
| Citrus | 5 | mg | 5.000 |
| naringinine | | | |
| Cocoa Extract | 200 | mg | 200.000 |
| Standardized for PEA | | | |
| (Phenylethylamine, | | | |
| tyramine and 10% | | | |
| theobromine) | | | |
| Green Tea Extract | 165 | mg | 165.000 |
| Standardized for ECGC | | | |
| (epigallocatechin gallate polyphenols | | | |
| and | | | |
| 40% caffeine) | | | |
| Hoodia gordonii cactus | 7.5 | mg | 7.500 |
| (whole plant/less roots) | | | |
| Plus suitable excipients | | | |

A formula suitable for use over extended periods of time (weeks or months) to reduce body mass is provided in Formula 9.

Formula 9

| | | | |
|---|---|---|---|
| Chromium (as chromium Chelavite ™ | 75 | mcg | 0.075 |
| dinicotinate glycinate) | | | |
| Vanadium (as vanadium amino acid | 15 | mcg | 0.015 |
| chelate) | | | |
| Glucomannan | 200 | mg | 200.000 |
| Sodium Carboxymethylcellulose | 50 | mg | 50.000 |
| Citrus naringinine | 5 | mg | 5.000 |
| Glucosamine HCL Or Glucosamine | 50 | mg | 50.000 |
| KCL | | | |
| Cocoa Extract Standardized for PEA | 162.500 | mg | 162.500 |
| (Phenylethylamine, tyramine and 10% | | | |
| theobromine) | | | |
| Green Tea Extract Standardized for | 125 | mg | 125.000 |
| ECGC (epigallocatechin gallate | | | |
| polyphenols and 40% caffeine) | | | |
| Hoodia gordonii cactus (whole | 100 | mg | 100.00 |
| plant/less roots) | | | |
| Plus suitable excipients | | | |

A formula suitable for use over extended periods of time (weeks or months) to reduce body mass is provided in Formula 10.

Formula 10

| | | | |
|---|---|---|---|
| Chromium (as chromium Chelavite ™ | 75 | mcg | 0.075 |
| dinicotinate glycinate) | | | |
| Vanadium (as vanadium amino acid | 15 | mcg | 0.015 |
| chelate) | | | |
| Glucomannan | 200 | mg | 200.000 |
| Sodium | 50 | mg | 50.000 |
| Carboxymethylcellulose | | | |
| Citrus | 5 | mg | 5.000 |
| naringinine | | | |
| Cocoa Extract Standardized for PEA | 162.500 | mg | 162.500 |
| (Phenylethylamine, tyramine and 10% | | | |
| theobromine) | | | |
| Green Tea Extract Standardized for ECGC | 125 | mg | 125.000 |
| (epigallocatechin gallate polyphenols and | | | |
| 40% caffeine) | | | |
| Hoodia gordonii cactus | 100 | mg | 100.00 |
| (whole plant/less roots) | | | |
| Plus suitable excipients | | | |

A potent formula suitable for use for rapid weight loss where obesity is serious, is provided in Formula 11.

Formula 11

| | | | |
|---|---|---|---|
| Chromium (as chromium Chelavite ™ | 0.075 | 50-200 | mcg |
| dinicotinate glycinate) | | | |
| Vanadium (as vanadium amino acid | 0.015 | 10-50 | mcg |
| chelate) | | | |
| Sodium | 50.000 | 0-200 | mg |
| Carboxymethylcellulose | | | |
| Citrus naringinine | | 0-15 | mg |
| Glucosamine HCL | | 0-100 | mg |
| Or Glucosamine KCL | | | |
| Cocoa Extract Standardized for PEA | 150.000 | 0-500 | mg |
| (Phenylethylamine, tyramine and 10% | | | |
| theobromine) | | | |
| Green Tea Extract Standardized for | 125.000 | 0-250 | mg |
| ECGC (epigallocatechin gallate | | | |
| polyphenols and 40% caffeine) | | | |
| Hoodia gordonii cactus | 100.000 | 5-200 | mg |
| (whole plant/less roots) | | | |
| 3-acetyl-7-oxo-dehydroepiandrosterone | 100.000 | 10-200 | mg |
| Ma Huang extract (15 mg ephedrine | 15.000 | 0-15 | mg |
| alkaloids) | | | |
| Plus suitable excipients | | | |

An alternative potent formula suitable for use for rapid weight loss where obesity is serious, is provided in Formula 12.

Formula 12

| | | | | |
|---|---|---|---|---|
| Chromium (as chromium Chelavite ™ | 75 | mcg | 50-200 | mcg |
| dinicotinate glycinate) | | | | |
| Vanadium (as vanadium amino acid | 15 | mcg | 10-50 | mcg |
| chelate) | | | | |
| Sodium Carboxymethylcellulose | 50 | mg | 0-200 | mg |
| Citrus Naringinine | 5 | mg | 0-15 | mg |
| Cocoa Extract Standardized for PEA | 150 | mg | 0-500 | mg |
| (Phenylethylamine, tyramine and 10% | | | | |
| theobromine) | | | | |
| Green Tea Extract Standardized for | 125 | mg | 0-250 | mg |
| ECGC (epigallocatechin gallate | | | | |
| polyphenols and 40% caffeine) | | | | |
| Hoodia gordonii cactus (whole plant/less | 100 | mg | 5-200 | mg |
| roots) | | | | |
| Coleus Forskohlii (10% Forskolin tuber) | 250 | mg | 0-250 | mg |
| Plus suitable excipients | | | | |

Without further elaboration, I believe that one of skill in the art can develop alternative formulations of hoodia gordonii cactus, alone or with caffeine and glucosamine, for long-term use to combat obesity and for weight loss. I accordingly intend that the scope of my patent be defined by the claims appended here, and not by the specific examples recited here. I intend the Abstract to be used for searching and classification, and not for claim interpretation. I intend the preamble of the claims to define and thus to limit the claim scope.

In the claims, I use the word “a” to include one or more (e.g., “a stimulant” means “one or more stimulants”).

1-7. (canceled)

8. A method of body weight reduction, comprising administering about 100 milligrams of hoodia gordonii, together with about 75 micrograms of chromium, about 15 micrograms of vanadium, about 50 milligrams of sodium carboxymethylcellulose, about 5 milligrams of citrus naringinine, and about 250 milligrams of green tea extract.

9. The method of claim 8, further comprising administering about 162.5 milligrams of cocoa PEA standardized extract.

10. The method of claim 9, comprising administering not less than about 100 milligrams of sodium carboxymethylcellulose, and not less than about 325 milligrams of cocoa PEA standardized extract.

11. The method of claim 8, further comprising administering, about 400 milligrams of glucomannan.

12. The method of claim 11, further comprising administering about 100 milligrams of glucosamine.

13. A method of weight reduction comprising administering about 7.5 milligrams of hoodia gordonii, together with about 75 micrograms of chromium, about 15 micrograms of vanadium, about 200 milligrams of glucomannan, about 50 milligrams of sodium carboxymethylcellulose, about 5 milligrams of citrus naringinine, about 200 milligrams of cocoa PEA standardized extract, and about 165 milligrams of green tea extract.

14. The method of claim 13, further comprising administering about 100 milligrams of glucosamine.

15. The method of claim 8, further comprising administering about 200 milligrams of glucomannan.

16. The method of claim 15, further comprising administering about 162.5 milligrams of cocoa PEA standardized extract, and about 125 milligrams of green tea extract.

17-40. (canceled)
